(19) United States
(12) Patent Application Publication
(10) Pub. No.: US 2018/0071228 A1
(43) Pub. Date: Mar. 15, 2018

(54) Trans-Dermal Vitamin Assembly

(71) Applicants: Adewale Oreshade, St. Louis, MO (US); Faidah Badru, St. Louis, MO (US)

(72) Inventors: Adewale Oreshade, St. Louis, MO (US); Faidah Badru, St. Louis, MO (US)

(21) Appl. No.: 15/266,226

(22) Filed: Sep. 15, 2016

(51) Int. Cl. A61K 9/70; A61K 31/519; A61K 33/26; A61K 33/06

(52) U.S. Cl. CPC A61K 9/7084; A61K 33/06; A61K 33/26; A61K 31/519

(57) ABSTRACT

A trans-dermal vitamin assembly for administering prenatal vitamins includes a patch. The patch is infused with prenatal vitamins. The patch is worn on skin. Thus, the prenatal vitamins are trans-dermally absorbed.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIE THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

### (2) Description of Related Art Including Information Disclosed Under 37 CFR 1.97 and 1.98

The disclosure and prior art relates to trans-dermal devices and more particularly pertains to a new trans-dermal device for administering prenatal vitamins.

### BRIEF SUMMARY OF THE INVENTION

An embodiment of the disclosure meets the needs presented above by generally comprising a patch. The patch is infused with prenatal vitamins. The patch is worn on skin. Thus, the prenatal vitamins are trans-dermally absorbed.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a top view of a trans-dermal vitamin assembly according to an embodiment of the disclosure.

FIG. 2 is a bottom view of an embodiment of the disclosure.

FIG. 3 is a cross sectional view taken along line 3-3 of FIG. 2 of an embodiment of the disclosure.

FIG. 4 is a front view of an embodiment of the disclosure in use.

### DETAILED DESCRIPTION OF THE INVENTION

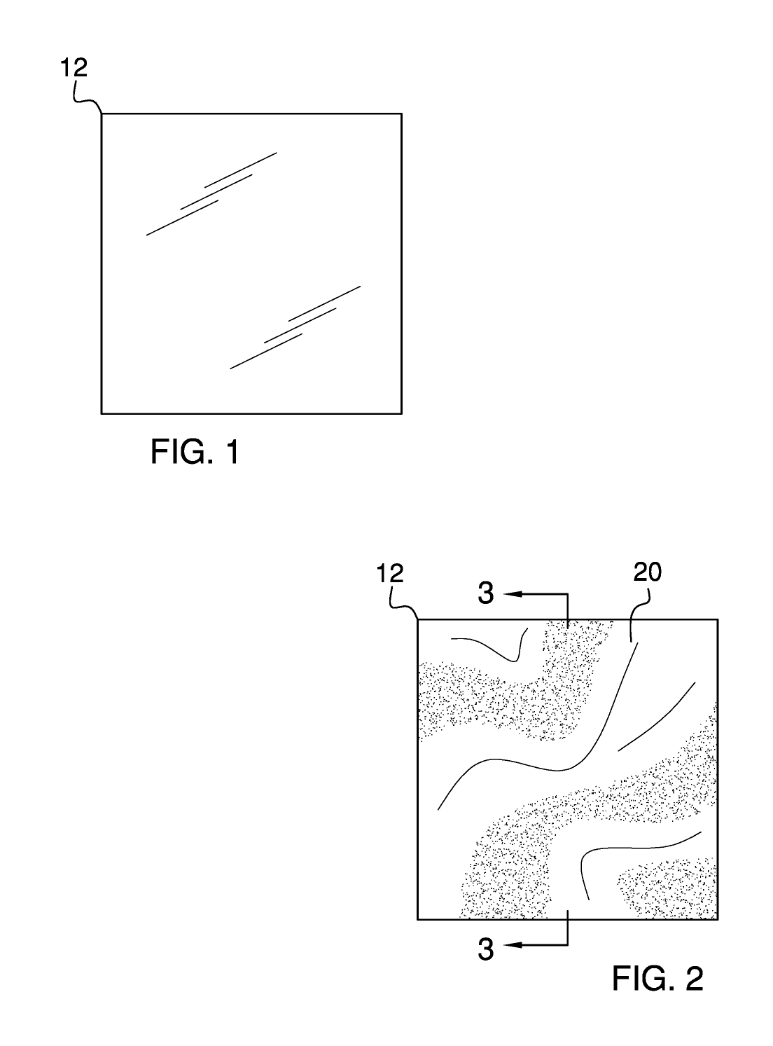

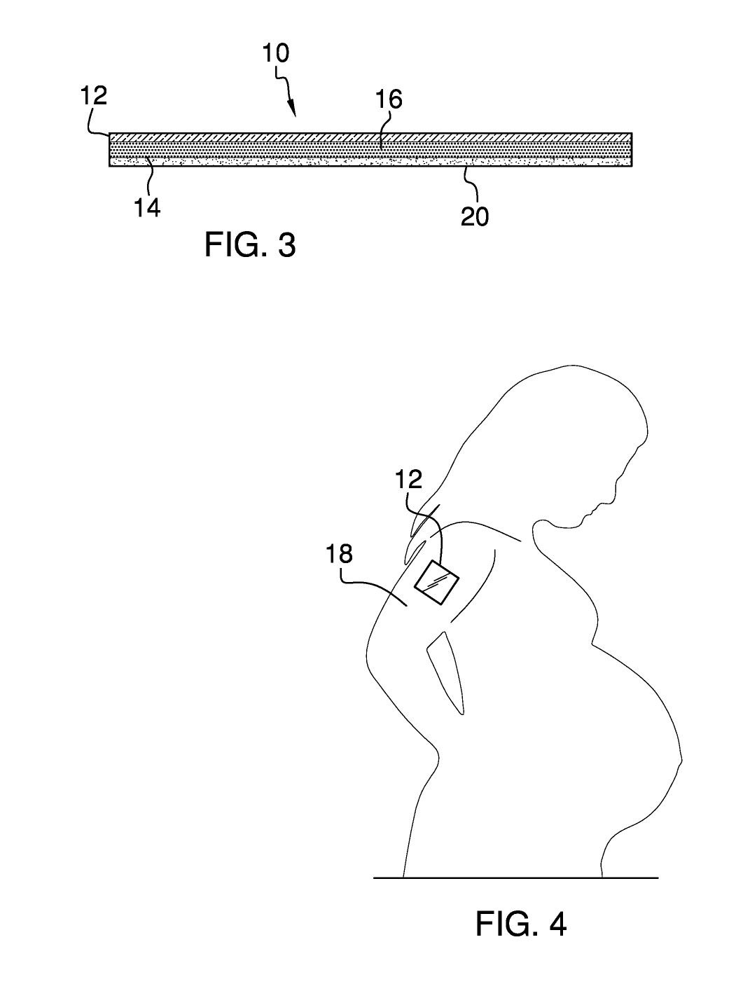

With reference now to the drawings, and in particular to FIGS. 1 through 4 thereof, a new trans-dermal device embodying the principles and concepts of an embodiment of the disclosure and generally designated by the reference numeral 10 will be described.

As best illustrated in FIGS. 1 through 4, the trans-dermal vitamin assembly 10 generally comprises a patch 12. The patch 12 is comprised of a fluid permeable material and the patch 12 has a first surface 14. The patch 12 is infused with prenatal vitamins 16. The prenatal vitamins 16 may be in fluid suspension. Moreover, the prenatal vitamins 16 may include folic acid, iron and calcium. The patch 12 may contain six hundred mcg of folic acid, twenty seven mg of iron and one thousand mg of calcium. The prenatal vitamins 16 may additionally include DHA, iodine, vitamin B6, vitamin B12, vitamin C, vitamin D, vitamin E and zinc.

The patch 12 is worn by a pregnant woman. The patch 12 is worn on skin 18 thereby facilitating trans-dermal absorption of the prenatal vitamins 16. Thus, the prenatal vitamins 16 are not orally administered. The patch 12 facilitates the prenatal vitamins 16 to be delivered when symptoms of morning sickness are present. Thus, the morning sickness symptoms do not inhibit the prenatal vitamins 16 from being administered.

An adhesive layer 20 is provided. The adhesive layer 20 is attached to the patch 12. The adhesive layer 20 adheres to the skin 18. Thus, the patch 12 is retained on the skin 18. The adhesive layer 20 is positioned on the first surface 14. The adhesive layer 20 completely covers the first surface 14.

In use, the patch 12 is manipulated such that the adhesive layer 20 engages the skin 18. Thus, the skin 18 absorbs the prenatal vitamins 16. The patch 12 delivers the prenatal vitamins 16 over a pre-determined amount of time. The patch 12 is removed from the skin 18 when the predetermined amount of time has elapsed. The patch 12 is continually worn during pregnancy. Thus, the prenatal vitamins 16 are administered without being affected by symptoms of morning sickness, including vomiting and nausea.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

1. (canceled)

2. (canceled)

3. (canceled)

4. A trans-dermal vitamin assembly being configured to deliver prenatal vitamins through skin thereby inhibiting nausea, said assembly comprising:
a patch being infused with prenatal vitamins, said prenatal vitamins being infused into said patch at a uniform depth across said patch, said patch being configured to be worn on skin thereby facilitating trans-dermal absorption of the prenatal vitamins, said patch having a first surface; and
an adhesive layer being attached to said patch wherein said adhesive layer is configured to adhere to the skin thereby facilitating said patch to be retained on the skin, said adhesive layer being positioned on said first surface, said adhesive layer completely covering said first surface.
